(19) United States
(12) Patent Application Publication
(10) Pub. No.: US 2009/0293772 A1
(43) Pub. Date: Dec. 3, 2009

(54) METHOD FOR STABILISING SOLUBLE METASTABLE SOLUBLE ANHYDRITE III, METHOD FOR PREPARING STABILISED SOLUBLE ANHYDRITE III HYDRAULIC BINDER, THE OBTAINED HYDRAULIC BINDER, USE OF THIS BINDER AND INDUSTRIAL FACILITY FOR CARRYING OUT SUCH A METHOD

(76) Inventors: Edouard Dumoulin, Nice (FR); Crisanto Palacios, Figueres (ES)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/095,777

(22) PCT Filed: Nov. 10, 2006
(86) PCT No.: PCT/EP2006/010828

§ 371 Date: Jan. 21, 2009

(30) Foreign Application Priority Data
Dec. 7, 2005 (IB) PCT IB2005/004006
Oct. 17, 2006 (EP) PCT EP06/10015

(51) Int. Cl. C04B 28/16; C01F 11/46; A61L 2/06

(52) U.S. Cl. 106/776; 423/555; 106/775; 422/307

(57) ABSTRACT

The invention relates to a method for stabilising a metastable soluble anhydrite III, to a method for producing a hydraulic binder based thereon, to the thus obtainable hydraulic binder, to a method for the use thereof in the cement industry and to an industrial plant for carrying out the inventive method. The method consists in stabilising a metastable soluble anhydrite III by mechanically stressing the particles thereof in such a way the crystal structure of the particles is modified and the metastable phase thereof is stabilised. Said invention makes it possible to stabilise the metastable soluble anhydrite III particles without using current steps for heating and quenching said particles.

The invention relates to a method for stabilizing soluble metastable anhydrite III as well as a method of preparing a stabilized soluble anhydrite III hydraulic binder.

It also relates to the hydraulic binder obtained as well as the use of this hydraulic binder in the cement industry.

It also relates to an industrial facility enabling the implementation of such a method.

The invention relates to the technical field of the cement industry and more particularly cement compositions resulting from dehydration of calcium sulfate.

Soluble anhydrite III hydraulic binders are well known to the person of skill in the art. A dehydration intensity—from 220° C. to 360° C.—of calcium sulfate, natural or synthetic (gypsum), having formula (CaSO₄, 2H₂O) or hemihydrate (plaster) having formula (CaSO₄, ½H₂O), results in the formation of soluble metastable anhydrite III having formula (CaSO₄, εH₂O) with ε from 0.1 to 0.2. An even more intense dehydration—from approximately 400° C.—results in the formation of anhydrite II (CaSO₄, 0H₂O), very weakly hygroscopic.

The soluble metastable anhydrite III being strongly hygroscopic, it rehydrates quickly into hemi-hydrate, or conventional calcium sulphate β, then returns to the calcium sulphate state according to the hygrometry of the air.

The person of skill in the art knows in particular via patents FR 2733496 (DUSSEL), FR 2767815 (COUTURIER), FR 2804423 (ENERGETIC INTERNATIONAL INDUSTRIES), WO 00/47531 (COUTURIER) or WO 2005/000766 (COUTURIER) of processes for preparation of stabilized soluble anhydrite III that include the following two successive steps:

a) curing a powdery composition, based on calcium sulfate (natural or synthetic gypsum or calcium sulfate) to form soluble metastable anhydrite III;

b) thermal quenching making it possible to stabilize the metastable phase of the anhydrite III.

The methods of the prior art thus teach the person of skill in the art to apply a thermal stress to anhydrite III particles in order to stabilize their metastable phase. This abrupt cooling is particularly important because it enables blocking and fixing of the crystalline structure of the anhydrite III particles in order to stabilize them.

This type of known method presents, however, a number of disadvantages. In fact, cooling is usually done by the injection of cold, dry air into the core of the material. It appears that the quality of stabilized anhydrite III particles is irregular, because the cooling is not effective on all of the particles.

Furthermore, a proportion of moist air present at the time of this cooling step leads to a rehydration of the metastable anhydrite III into calcium sulfate hemihydrate so that the proportion of industrially produced stabilized anhydrite III is not very high, absent a complex and costly facility.

Also, the heating is usually performed in rotary kilns requiring a substantial amount of energy to operate. Furthermore, these rotary kilns have a strong inertia, ie it requires a substantial amount of time to cool them or to make them attain the desired temperature. For these reasons, it is difficult and costly, in time and energy, to halt production.

Given the disadvantages of the prior art, the principal technical problem that the invention aims to resolve is to effectively stabilize soluble metastable anhydrite III particles, without resorting to cooling of the aforementioned particles.

An object of the invention is also to provide a method enabling preparation of stable anhydrite III particles, simple to implement and not requiring much energy.

Another object of the invention is to provide a simple and inexpensive industrial facility to enable implementation of this method.

The invention has another object of providing an anhydrite III hydraulic binder having good mechanical performance.

To solve these technical problems, the applicant has now demonstrated that the application of a mechanical stress to metastable anhydrite III particles effectively stabilizes the aforementioned particles.

Within the meaning of this invention, “stable” means the fact that the rehydration kinetics of the anhydrite III particle is strongly slowed. In this way, the hydraulic binder obtained can be stored and conserved a long time without particular constraint, its properties remaining almost constant over time.

The document MURAT, M.; EL HAJJOUJI, A. “Activation of solids by mechanical grinding, consequences for calorimetric investigation on the hydration rate orthorhombic anhydrite.” THERMOCHIMICA ACTA, Vol. 85, 1985, pages 119-122, describes a method to improve the hydration speed of anhydrite orthombique, including:

a) the calcination of gypsum powder for 5 hours at 750° C. to obtain synthetic orthorhombic CaSO₄ anhydrite with many superficial defects,

b) after cooling, the anhydrite is mechanically micronized in a Fritsch centrifugal grinder for a period of 4 to 120 min.

This grinding improves the reactivity of the new surface by the local introduction of stresses and defects that behave as preferential sites in a chemical reaction. The new surface is very sensitive to water vapor. However, this activation does not provide the advantage of stabilizing the metastable phase of the anhydrite III.

The solution provided by the invention includes applying a mechanical stress to soluble metastable anhydrite III particles in order to stabilize their metastable phase. This method, inexpensive in energy, enables effective stabilization of the anhydrite III particles by altering their crystalline structure.

According to an advantageous feature of the invention enabling application, in a simple manner, of a mechanical stress to soluble metastable anhydrite III particles, these particles are impacted against a wall. And preferentially these soluble metastable anhydrite III particles are injected into an impacting conduit configured so that the aforementioned particles impact its walls during their travel.

According to another advantageous feature of the invention enabling application of an optimal mechanical stress to the soluble metastable anhydrite III particles, these particles are impacted at a velocity between 5 m/s and 30 m/s.

The invention also relates to a method of preparing an anhydrite III hydraulic binder, characterized by the fact that:

a) a powdery calcium sulfate composition is heated to form soluble metastable anhydrite III,

b) a mechanical stress is applied to the soluble metastable anhydrite III particles in order to stabilize their metastable phase.

This method enables stabilization of the calcium sulfate particles in soluble metastable anhydrite III phases by a mechanical stress. The hydraulic binder obtained by this method has a good moisture resistance and its rehydration in the air is slowed down. Moreover, physical and mechanical performance of concrete or mortar type products obtained by using this binder are at least as good as those of products obtained by the use of similar hydraulic binders known to the person of skill in the art.

According to yet another advantageous feature of the invention, enabling micronization of the anhydrite III particles having large diameter before stressing them mechanically, the powder composition is heated in order to vaporize H₂O molecules contained in the calcium sulfate particles and cause the break up of the particles. And preferably, the calcium sulfate powder composition is heated by a flash method at a temperature between 400° C. and 700° C. and in an atmosphere saturated with water vapor.

According to yet another advantageous feature of the invention enabling simplification of the preparation of the hydraulic binder, the steps a) and b) are performed simultaneously by injecting the powder composition into a stream of warm air saturated with water vapor and having a temperature between 400° C. and 700° C., the aforementioned flow of hot air traversing the impacting conduit. In this way, calcium sulfate particles simultaneously undergo a thermal stress, which has the effect of breaking them up and creating soluble metastable anhydrite III, and a mechanical stress, which has the effect of stabilizing the metastable phase of the latter.

According to yet another advantageous feature of the invention, thermal quenching is performed on the particles obtained after step b).

According to an advantageous feature of the invention enabling variation of the physical and mechanical properties of the hydraulic binder the temperature and heating time of the calcium sulfate powder composition are regulated in order to form soluble metastable anhydrite III and/or anhydrite II and/or β hemihydrate of calcium sulphate. “Anhydrite III and/or anhydrite II and/or β hemihydrate of calcium sulphate” should be understood as meaning “soluble metastable anhydrite III alone” or “soluble metastable anhydrite III and anhydrite II” or “soluble metastable anhydrite III and β hemihydrate of calcium sulphate” or “soluble metastable anhydrite III and anhydrite II and β hemihydrate of calcium sulphate.”

To avoid having the soluble metastable anhydrite III particles rehydratate too quickly before step b), at step a) the temperature and heating time of the calcium sulfate powder composition are regulated to form particles having soluble metastable anhydrite III at the core and anhydrite II at the surface.

According to a preferred implementation feature, a powder composition is heated, the powder composition being based on natural gypsum, synthetic gypsum, or hemihydrate of calcium sulphate.

To improve the properties of the hydraulic binder, the powder composition is mixed with one or more compounds from the following list: lime, hydroxide of lime, marble powder, calcium carbonate, polycarboxylate.

Given the remarkable properties observed by the applicant, an object of the invention is also the hydraulic binder obtained by the method described above, the aforementioned binder being useable for the preparation of concrete or mortar type material.

Another object of the invention is an industrial facility for the implementation of the method described above, the aforementioned facility having a means for heating the calcium sulfate powder composition and forming soluble metastable anhydrite III and a means for applying a mechanical stress to the aforementioned particles in order to stabilize their metastable phase.

According to an advantageous feature of the invention simplifying the design and implementation of the method, soluble metastable anhydrite III particles are injected into an impacting conduit configured so that the aforementioned particles impact its walls during their travel, the aforementioned conduit being connected to a hot air generator. And to increase the impacting regions, the conduit preferentially has a substantially toroidal shape.

According to yet another advantageous feature of the invention, in order to avoid having the anhydrite III particles rehydrate too quickly at the outlet of the impacting conduit, the outlet is coupled to a means for separating the water vapor from the solid particles. And to increase the profitability of the facility, the water vapor is preferentially directed towards a filter designed to recover fine residual particles.

According to yet another advantageous feature of the invention enabling optimization of their stabilization and their micronization, the particles exiting the impacting conduit may be directed to a second impacting conduit connected to a compressed air source.

According to yet another advantageous feature of the invention, a thermal quenching device is positioned downstream from the first and/or second impacting conduit.

To prevent any entry of exterior moist air, the facility optimally includes a pressurization device arranged so as to create an overpressure in the aforementioned facility.

In an implemention variantion, the means for applying a mechanical stress to the soluble metastable anhydrite III particles, can be a piston arranged so as to apply a mechanical force on the aforementioned particles.

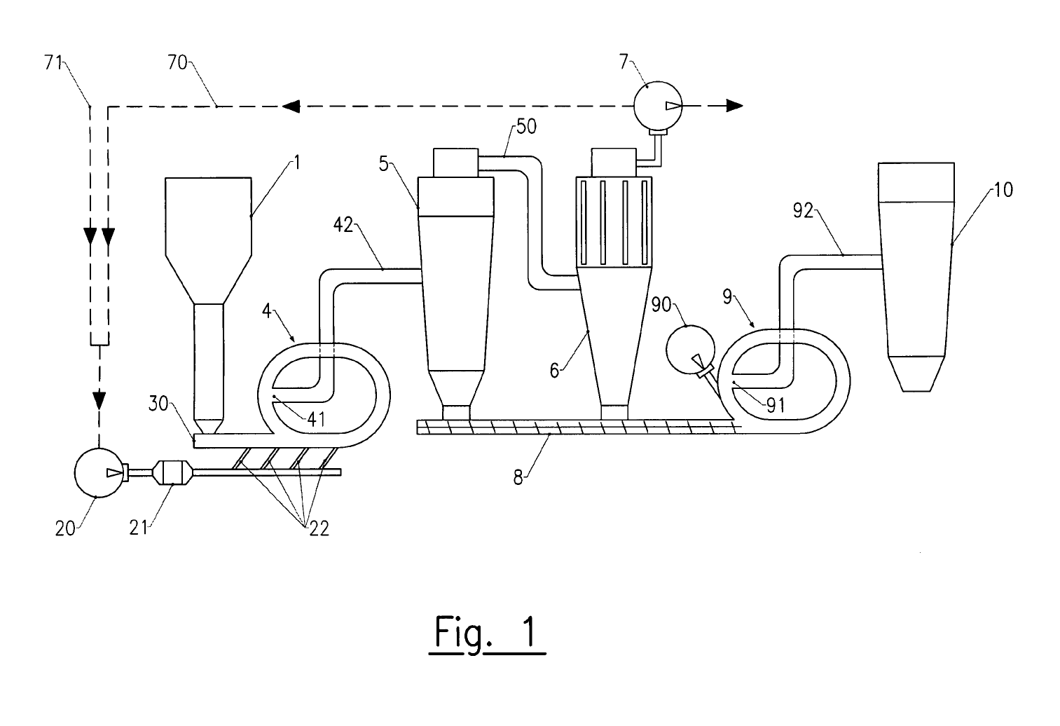

Other features and advantages of this invention will better reemerge at the reading of the description that will follow, made by way of guiding non-limiting example, with regard to the attached drawing on which FIG. 1 schematically represents a preferred implementation mode of the facility object of the invention.

In referring to the attached figure, a calcium sulfate powder composition is stored beforehand in a silo 1. The powder composition employed is optimally based on natural gypsum, synthetic gypsum (including sulphogypsum, phosphogypsum, borogypse, titanogypsum) or hemihydrate (α or β) of calcium sulphate.

The powder composition can be mixed with one or more compounds from the following list: air-slaked lime, hydraulic lime, marble powder, calcium carbonate, polycarboxylate. These complementary additives known to the person of skill in the art enable improvement of the hydraulic binder properties and particularly mechanical resistances to compression, fire rating, etc. In practice, optimally the soluble metastable anhydrite III particles and/or anhydrite II and/or β hemihydrate of calcium sulphate between 1% and 15% by weight is mixed with lime or hydroxide of lime. This post calcination mixture aims to improve the physical-chemical reaction that occurs later in the method.

The powder composition may also be mixed with quicklime in order to capture the residual moisture and/or moisture from the ambient air to slow rehydration of the anhydrite III.

The particle size of the powder composition to be treated is between 20 μm and 15 mm depending on the nature of calcium sulfate used (natural, synthetic or hemihydrate).

The powder composition is heated in a heating device so as in order to form only soluble metastable anhydrite III particles or particles associated with particles of anhydrite II and/or particles of calcium sulfate β hemihydrate. The presence of anhydrite II and/or β hemihydrate of calcium sulfate enables modification of the physical and mechanical properties of the hydraulic binder object of the invention.

The anhydrite II/anhydrite III_{soluble} weight ratio is preferentially between 1% and 100%, depending on the applications of hydraulic binder object of the invention. For example, a binder having a anhydrite II/anhydrite III_{soluble} weight ratio between 20% and 40% will have good mechanical properties.

This powder composition is heated between 180° C. and 700° C. for a time ranging from a few seconds to several hours. The temperature and heating time depend on several factors including principally the particle size, the type of powder composition to be treated and the heating method used. The heating may be performed directly or indirectly, by flash calcination methods, rotary kilns, baking cauldrons or any equivalent calcination device.

The regulation of the different calcination parameters enables adjustment of the proportion of soluble metastable anhydrite III and/or anhydrite II and/or β hemihydrate of calcium sulphate according to the characteristics of the final composition sought.

According to a preferred feature of the invention, the calcium sulfate powder composition is heated in order to vaporize H₂O molecules contained in the particles of calcium sulfate and cause the breakup of the latter. To carry this out, implementation of the flash method described below is preferred, but any other method enabling attainment of this result can be used by the person of skill in the art.

The preferred heating device is optimally a calcinator constituted by an air turbine 20 associated with a burner 21. The powder composition is injected into a duct 30 arranged with hot air injectors 22 and is transported at high velocity (between 5 m/s and 30 m/s) by the flow of hot air thus generated. The injectors 22 are configured to create turbulence and promote thermal exchanges.

The flash to the calcium sulfate particles, already micronized (maximum diameter of 1 mm), can be performed at a temperature between 280° C. and 320° C. for about 5 seconds, so as not to overbake the anhydrite III particles.

According to a preferred feature of the invention, the flash is performed in an atmosphere saturated with water vapor and at a temperature between 400° C. and 600° C., preferably 500° C. These high temperatures enable vaporization of the H₂O molecules contained in the calcium sulfate particles, which has the effect of breaking up the particles and reducing their diameter. It is thus possible to treat particles of several millimetres in diameter (up to 15 mm) and to reduce their diameter by half before mechanically stressing them. The atmosphere saturated with water vapor enables, even at temperatures on the order of 500° C., formation of soluble metastable anhydrite III particles without overbaking.

By adjusting the flow rate of the hot air flow generated by the flash calcinator, the heating temperature and particle diameter of the powder composition, the person of skill in the art can vary the amount of soluble metastable anhydrite III and/or anhydrite II and/or β hemihydrate of calcium sulphate. For example, a stream of warm air of 500° C., having a velocity of 5 m/s enables treatment of a calcium sulfate composition having a particle size on the order 10 mm, to form between 60% and 80% of soluble metastable anhydrite III and between 20% and 40% of anhydrite II.

To avoid the rehydration of the anhydrite III particles in metastable phase in case of introduction of moist exterior air, one can also regulate the different calcinations parameters to form particles with having soluble metastable anhydrite III at the core and anhydrite II at the surface. Since anhydrite II is weakly hygroscopic, soluble metastable anhydrite III remains protected by the envelope of anhydrite II.

Other methods to remove H₂O molecules contained in the calcium sulfate particles to form anhydrite III may be employed. One could for example foresee using centrifuge methods or using ultra-sound.

In accordance with the invention, a mechanical stress is applied to anhydrite III particles in order to stabilize their metastable phase. It was demonstrated that this mechanical stress enables modification of the crystalline structure of anhydrite III particles and/or anhydrite II and/or β hemihydrate of calcium sulphate, particularly by densifying them, and obtaining higher mechanical resistances and substantially reducing the metastability, ie the capacity to reabsorb water.

This modification of the crystalline structure is due to the collision and friction of the particles themselves, as will as a modification of the surface energy of the aforementioned particles. It is believed that under the effect of the mechanical stress, the crystalline structure is distorted so there is more space available for the return of H₂O molecules.

The application of the mechanical stress is preferably carried out by impacting soluble metastable anhydrite III particles (and/or anhydrite II and/or β hemihydrate of calcium sulphate) against a wall. However, other equivalent methods enabling application of a mechanical stress can be employed. One could for example employ a piston arranged so as to apply a mechanical force on the particles, the latter being crushed by the piston.

The application of this mechanical stress also enables associating the phase III anhydrite with the anhydrite phases 11 and/or β hemihydrate of calcium sulphate to form a new type of hydraulic binder. On could also foresee applying a mechanical stress directly to a mixture including soluble stabilized anhydrite III (for example Gypcement®), anhydrite II, and/or β hemihydrate of calcium sulfate to obtain a hydraulic binder including particles whose crystalline structure includes soluble anhydrite III phases associated with anhydrite phases II.

Referring to FIG. 1, the duct 30 is coupled to an impacting conduit 4 configured so that the soluble metastable anhydrite III particles impact its walls during their travel. The particles are projected at a velocity between 5 m/s and 30 m/s against the wall, the velocity inducing a stabilization depending on the size and nature of the particles to be stabilized. The air turbine 20 associated with the burner 21 can generate a stream of warm air having such velocity. The synthesis of anhydrite III particles (and/or anhydrite II and/or β hemihydrate of calcium sulphate) by joint action of thermal shock at very high temperature and mechanical shocks at very high velocities ensures cohesion of the hydraulic binder.

The impacting conduit 4 optimally has a substantially toroidal shape so that, at each change of direction, the particles impact the walls. The impacting conduit 4 can be perfectly toroidal or include straight portions before the changing of direction. However, the impacting conduit 4 may have any other configuration enabling the particles to impact on the walls, for example, conduits having a ‘L’ or ‘U’ form. In practice, we prefer to use a turbo-dryer RINA-JET® manufactured by the company RIERA NADEU SA.

By impacting on the walls, the anhydrite III particles (and/or anhydrite II and/or β hemihydrate of calcium sulphate) will not only stabilize, but also break up, enabling micronization of the aforementioned particles and reducing the particle the size between 5 μm and 50 μm.

If for the heating device the calcination parameters are regulated to form only soluble metastable anhydrite III particles (possibly enveloped in a layer of anhydrite II), a device for introducing anhydrite II and/or β hemihydrate of calcium sulfate (not shown) can be positioned after, or optimally before, the first impacting conduit 4.

The facility shown in FIG. 1 enables concurrent excertion, on the particles of the powder composition, of:

a thermal shock from the flow of hot air generated by the air turbine 20 and the burner 21,

a mechanical stress due to the impact of the particles on the walls of the impacting conduit 4.

However, it is possible to apply a mechanical stress to unheated soluble metastable anhydrite III particles (and/or anhydrite II and/or β hemihydrate of calcium sulphate) previously stored at ambient temperature. It is also possible to apply a mechanical stress to already stabilized anhydrite III particles in order to increase the mechanical properties of the hydraulic binder.

The step of application of the mechanical stress accomplishes completion of the standard stabilization process of anhydrite III. This step can be repeated successively over time, at higher or lower temperatures, in order to improve certain physical and mechanical qualities of the hydraulic binder, such as the anhydrite III/anhydrite II weight ratio, the stability of the binder to the absorption of water, the rehydration kinetic, and so on. This technology offers at several levels the possibility to precisely regulate the required parameters for the hydraulic binder, and manage crystallographic phenomena of the anhydrite III phases (and/or anhydrite II and/or β hemihydrate of calcium sulphate).

In referring to FIG. 1, the outlet 41 of the impacting conduit 4 is positioned on the inner side of the conduit. This arrangement enables recovery of only the particles that have attained the desired diameter. As a result of centrifugal accelerations generated in the conduit 4, particles having large diameter and therefore having high weight, are attracted toward the exterior wall of the conduit against which they break up and micronize. Only the particles having small diameter and low weight can reach outlet 41 and be recovered. As long as the particles are not micronised to desired diameter, they can not reach the outlet 41 and continue to move in the conduit 4.

In accordance with the facility shown on the attached figure, the outlet 41 of the centrifugal conduit 4 is coupled via a conduit 42, with a means 5 for separating the water vapor from the solid particles. In practice, this relates to a filter cyclone in which the solid particles are directed towards the bottom and the water vapor towards the top.

Optimally, recovered water vapor is directed, via a conduit 50, to a second filter 6 to recover fine residual particles. The second filter 6 is connected to a water vapor extracting device 7, of the air pump type.

In order to improve the energy efficiency of the facility, it is possible to feed the air turbine 20 via lot air 70 from the water vapor extraction device 7 mixed with fresh air 71.

The solid particles from the impacting conduit 4 and/or means 5 for separating the water vapor from the solid particles and/or from the second filter 6 can be transported via a conduit to transport 8, via an Archimedes screw, to a second impacting conduit 9 connected to a compressed air source 90. The second impacting conduit 9 is similar to that described above and operates the same way. Any other device capable of applying a mechanical stress to the particles can be used by the person of skill in the art.

The compressed air enables placing the particles of the hydraulic binder into circulation in the second conduit 9 so that they can impact the walls of the latter at an appropriate velocity. Cold compressed air, having high-pressure ranging from 2 bars to 15 bars, is injected. This mechanical stress completes the breakups of the particles to reduce the size between 1 μm and 10 μm.

The hydraulic binder returning in the second impacting conduit 9 is at a temperature lower than 120° C. because of successive thermal exchanges via contact with different apparatus. However, by insulating these apparatus, it is possible to maintain a hydraulic binder at a temperature on the order of 300° C. The contact of the hot particles with compressed cold air acts as a thermal quenching and completes the stabilization of anhydrite III particles. Any other thermal quenching device known to person of skill in the art can be positioned downstream from the second impacting conduit 9 or first impacting conduit 4.

Referring to FIG. 1, the outlet 91 of the second impacting conduit 9 is coupled via a conduit 92 to a reservoir 10, enabling storage of the hydraulic binder before its conditioning.

In an implemention variantion not show, the outlet 91 of the second impacting conduit 9 is coupled to a third impacting conduit and so on until acquisition of a hydraulic binder having attained the sought characteristics.

In an implemention variantion not show, the solid particles from the impacting conduit 4 and/or from the means 5 for separating the water vapor from the solid particles and/or from the second filter 6, are transported to a flash calcination device having a straight conduit. Having a straight conduit enables effective association of the particles with other minerals materials (air-slaked lime, hydraulic lime, quick lime, marble powder, calcium carbonate, polycarboxylate, . . . ). Moreover, with a second flash method, the thermal treatment of the anhydrite III is completed and the calcination parameters are regulated so as in order to form particles having anhydrite III at the core and anhydrite II at the surface.

It is advantageous to maintain a dry atmosphere throughout the facility (humidity of the air less than 10%, preferably between 0 and 5%) from the outlet of storage silo 1 up to the reservoir 10. To control this humidity, a pressure boosting device is employed to avoid introduction of moist air outside. This pressure boosting device includes a dry air compressor arranged with humidity sensors in order to pressurize the transport conduits and the entire facility. Any other equivalent pressure boosting device expedient for the person of skill in the art may be employed.

To maintain a dry atmosphere throughout the facility object of the invention, dehumidifiers layed out with humidity controllers may also employed.

The hydraulic binder obtained possesses quite remarkable characteristics:

stability to moisture and reabsorption of water (less than 2%),

high particle density,

high solubility,

high mechanical resistance in association with any form of aggregates: Rc ranging from 40 MPa to 80 Mpa and Rf ranging from 10 to 20 Mpa Mpa.

very low porosity related to the fineness of the binder for the manufacture of overdensified materials,

increased adherent performance on any type of support, compatibility with water reducing additives, producing high performance composite technologies,

exceptional aesthetic qualities resulting from the fineness and the density of materials obtained via the aggregates considered,

improvement of the fire behavior of the compositions developed from the aforementioned hydraulic binder.

The hydraulic binder obtained can be used in the preparation of a concrete or mortar type material. The applicant found experimentally that by combining the hydraulic binder obtained in accordance with to the invention, with cement, for example of the Portland or calcium hydroxide (lime) type, the material obtained was water resistant and had improved mechanical performance, especially when 70% to 90% p/pₘᵢₓₜᵤᵣₑ hydraulic binder object of the invention is mixed with 10% to 30% p/pₘᵢₓₜᵤᵣₑ of cement. The mechanical performance are increased by 10% to 15%.

The applicant also found that adding 5% p/pₘᵢₓₜᵤᵣₑ lime, to the hydraulic binder object of the invention, enables fluidization of the reference mortar and augmentation of mechanical resistances by 30%.

1-24. (canceled)

25. A method to stabilize soluble metastable anhydrite III, the method comprising applying a mechanical stress to soluble metastable anhydrite III particles in order to stabilize their crystalline structure and stabilize their metastable phase, the mechanical stress being applied by impacting the soluble metastable anhydrite III particles against a wall.

26. The method according to claim 25 further including injecting soluble metastable anhydrite III particles into a conduit configured so that the particles impact its walls during their travel.

27. The method according to claim 26 further including impacting the soluble metastable anhydrite III particles at a velocity between 5 m/s and 30 m/s.

28. A method of preparing an anhydrite III hydraulic binder, the method comprising:
a) heating a powdery calcium sulfate composition to form soluble metastable anhydrite III, and
b) applying a mechanical stress to the soluble metastable anhydrite III particles in order to stabilize their metastable phase, the mechanical stress being applied by impacting the soluble metastable anhydrite III particles against a wall.

29. The method according to claim 28 further including heating the calcium sulfate powder composition in order to vaporize H₂O molecules contained in the particles of calcium sulfate and cause the breakup of the latter.

30. The method according to claim 28 further including heating the calcium sulfate powder composition by a flash method at a temperature between 400° C. and 700° C., in an atmosphere saturated with water vapor.

31. The method according to claim 28 further including performing steps a) and b) simultaneously by injecting the powder composition into a stream of warm air saturated with water vapor and having a temperature between 400° C. and 700° C., the flow of hot air traversing the impacting conduit.

32. The method according to claim 28 further including performing thermal quenching on the particles obtained after step b).

33. The method according to claim 28 further including regulating the temperature and heating time of the calcium sulfate powder composition in order to form soluble metastable anhydrite III and/or anhydrite II and/or β hemihydrate of calcium sulphate.

34. The method according to claim 28 further including regulating the temperature and heating time of the calcium sulfate powder composition in order to form particles having soluble metastable anhydrite III at the core and anhydrite II at the surface.

35. The method according to claim 28 wherein step a) includes heating a powder composition, the powder composition being based on natural gypsum, synthetic gypsum, or hemihydrate of calcium sulphate.

36. The method according to claim 35 further including mixing the powder composition with one or more compounds from the following list: air-slaked lime, hydraulic lime, quick lime, marble powder, calcium carbonate, polycarboxylate.

37. A hydraulic binder including soluble stabilized anhydrite III, characterized by the fact that it is obtained by the method in accordance with claim 28.

38. Using a hydraulic binder in accordance with claim 37 for the preparation of a concrete or mortar type material.

39. A facility for the implementation of the method in accordance with claim 28, the facility including a means for heating the calcium sulfate powder composition and forming soluble metastable anhydrite III and a means for applying a mechanical stress to the particles in order to stabilize their metastable phase, the soluble metastable anhydrite III particles being injected into a conduit configured so that the particles impact its walls during their travel, the conduit being coupled to a hot air generator.

40. The facility according to claim 39 wherein the impacting conduit has a substantially toroidal shape.

41. The facility according to claim 39 wherein the outlet of the centrifugal conduit is coupled to a means for separating the water vapor from the solid particles.

42. The facility according to claim 41 wherein the water vapor is directed towards a filter designed to recover fine residual particles.

43. The facility according to claim 39 wherein the particles exiting the impacting conduit are directed to a second impacting conduit coupled to a compressed air source.

44. The facility according to claim 39 wherein a thermal quenching device is positioned downstream from the first and/or second impacting conduit.

45. The facility according to claim 39 further including a pressurization device arranged so as to create an overpressure in the facility.
